(19) United States
(12) Patent Application Publication
(10) Pub. No.: US 2004/0147985 A1
(43) Pub. Date: Jul. 29, 2004

(54) Dermatological treatment flashlamp device and method

(75) Inventors: Dean A. MacFarland, Magnolia, MA (US); David A. Gollnick, San Francisco, CA (US); Greg J.R. Spooner, Kensington, CA (US); Kevin P. Connors, San Francisco, CA (US)

Correspondence Address: HAYNES BEFFEL & WOLFELD LLP, P O BOX 366, HALF MOON BAY, CA, 94019, US

(73) Assignee: Altus Medical, Inc., Burlingame, CA

(21) Appl. No.: 10/351,981

(22) Filed: Jan. 27, 2003

(51) Int. Cl. A61B 18/18

(52) U.S. Cl. 607/90; 607/88; 606/9

(57) ABSTRACT

A power supply comprising a chopper circuit with an inductive filter element may drive a flashlamp to direct flashlamp radiation to a patient's skin. The waveform may have a generally constant current value and may be substantially independent of pulse width repetition rate and of pulse repetition rate. The flashlamp may be selected according to the type of treatment and the expected width of the treatment area. The wavelength of the radiation to be directed to a patient may be limited to a shallow tissue-penetrating, strongly melanin-absorbing wavelength spectrum, such as at most about 590 to 850 nm or at most about 590 to 700 nm. The chosen wavelength spectrum may be-within the UVA through UVB wavelength spectrum so to cause localized pigmentation in a patient's skin. The chosen wavelength spectrum may be a continuous wavelength spectrum. A handpiece may have a housing comprising a housing interior with the flashlamp so that light from the flashlamp passes through the housing interior and out of an aperture for dermatological treatment of a patient. The flashlamp arc length is preferably about 20% greater than the aperture length.

### BACKGROUND OF THE INVENTION

High-intensity light is applied to skin for various medical treatments. Two of the most common sources of light used for epidermal treatments are lasers and flashlamps. Flashlamps are commonly used to remove hair and for treatment of microvascular abnormalities and other related conditions in the skin.

The light output of plasma discharge flashlamps is divergent, large in area, broadband and incoherent in character. Such lamps can produce relatively large peak powers over the UVA-NIR optical range, provided that sufficiently high electrical currents can be driven through them. The optical output may be directed towards skin, using appropriate optics and wavelength filters appropriately designed to couple the desired optical bands into skin. Presently, a number of manufacturers of flashlamp medical devices for dermatology use a reservoir-discharge circuit (RDC) or a pulse-forming network (PFN) to drive xenon or krypton flashlamps.

In an RDC design, achieving high currents for reasonably long pulse widths requires charging the reservoir capacitance bank to a high voltage and essentially dropping the bank voltage across a flashlamp. For this to be an effective method of energy delivery, the arc length must be longer than reasonable treatment apertures or the capacitor bank must be very large.

Longer arc lengths mean that less of the total lamp optical output can be collected into a reasonable treatment spot size. This has two consequences: (1) reduced electrical-to-optical efficiency and (2) increased handpiece size to accommodate the longer lamp. The second consequence is serious as the form factor of the lamp and the incoherence and divergence of the light generally makes the mechanical/ergonomic aspects of the handpiece design the major weakness in using a flashlamp dermatology system.

Another limitation is that in order to maintain useful optical output peak powers over the entire optical pulse width, the energy storage capacitor must store much more energy than is actually used in the treatment pulse. This is because the voltage necessarily falls as the capacitor bank discharges energy into the flashlamp, and the optical power out of the lamp varies as the third power of the lamp voltage drop. Practical-sized capacitor banks are quickly depleted during a pulse or series of pulses, resulting in large drops in the optical power delivered to skin. PFN circuits typically include a capacitor and an inductor in series with the flashlamp. PFN circuits are impedance-matched for a particular lamp impedance. PFNs do not suffer from the disadvantages associated with RDC's (see above section) but they do have the limitation of being limited to a single pulse width. PFN driven lamps are less effective as a tool for selective photothermolysis, as different targets in the skin have different thermal relaxation times and require various optical pulse widths for effective treatment. In response to this limitation, multiple PFNs or portions thereof can be connected to a single lamp. These can be used in ways to create multiple pulses in rapid succession, or to switch from longer to shorter pulse widths. However, due to the cost of the switches and energy storage elements required to achieve variability of the output pulse, this is typically not considered an economical approach.

A number of disease, medical or trauma conditions give rise to cosmetically undesirable pigmentary variation in human skin. Scars, temporary or permanent hypo- and hyper-pigmentation, striae distensae (stretch marks), leukoderma, poikiloderma of Civatte, etc., are examples of conditions in which a melanin pigmentation cosmetic defect is presented by at least one component of the condition. Dermatologists and suffers of these conditions use a variety of approaches to reduce the contrast between pigment variation regions. Existing approaches employ two general methods: I. removal of abnormally pigmented skin, or a component of same, with the goal of new growth that contains cosmetically desirable “natural” pigmentation (examples include ablative laser skin such as resurfacing, chemical peels and dermabrasion, and specific targeting of melanin-bearing tissue through selective absorption of specific light wavelengths as in the use of Q-switched 532 nm lasers), or II. treatment of skin with UV light sources to promote the formation of melanin in melanin-deficient skin (for example, UV lamps and excimer laser therapies). Problems associated with these treatments include: a) resistance of pigment-deficient areas to melanogenesis or pigment induction (associated with II.), b) overtreatment or loss of desired pigmentation (associated with I.), c) lack of spatial localization of treatment (associated with II.), d) lack of control of pigment induction e) thermal injury or scarring (typically associated with I).

See the following U.S. patents: U.S. Pat. Nos. 5,282,842; 5,320,618; 5,626,631; 5,683,380; 5,830,208; 5,849,029; 5,964,749; 6,214,034 and 6,383,176.

### SUMMARY OF THE INVENTION

A first aspect of the invention is directed to a method for driving a dermatological treatment flashlamp. A power supply, comprising a chopper circuit with an inductive filter element, and a desired treatment waveform are selected. A flashlamp is supplied with electrical current from the power supply to create the desired treatment waveform. At least one pulse of radiation is directed from the flashlamp to a patient. The power supply preferably operates in a pulse width modulation (PWM) controlled current mode or a PWM controlled power mode. The method may comprise determining an expected type of treatment, and then selecting a flashlamp type according to the type of treatment. The method may also comprise determining an expected width of a treatment area, and then selecting a flashlamp having a flashlamp arc length corresponding to the expected width. The wavelength of the radiation to be directed to a patient may be limited to a shallow tissue-penetrating, strongly melanin-absorbing wavelength spectral region in which the selectivity of melanin absorption over other chromophores such as hemoglobin is advantageous, such as at most about 590 to 850 nm or at most about 590 to 700 nm. The chosen wavelength spectrum may be a continuous wavelength spectrum.

A second aspect of the invention is directed to a method for enhancing the operation of a dermatological treatment flashlamp device. A power supply, comprising a chopper circuit with an inductive filter element, is selected. The power supply is operably coupled to a flashlamp of a dermatological-treatment flashlight device so that electrical current from the power supply may be supplied to the flashlamp, whereby at least one pulse of radiation from the flashlamp may be directed to a patient. A desired current waveform may be selected so that electrical current from the power supply having the desired waveform may be supplied to the flashlamp. The current waveform may comprise a pulse train of a chosen set of fixed or varying amplitudes.

A third aspect of the invention is directed to a method for enhancing the operation of a pigmented lesion treatment flashlamp device. A power supply, comprising a chopper circuit with inductive filter element, is selected. A treatment area width is determined. A flashlamp, having a chosen flashlamp arc length, is selected. The flashlamp arc length is chosen to be about 20% longer than the treatment area width. The wavelength of the radiation to be directed to a patient is limited to a wavelength spectrum of above about 590 nm. The power supply is operably coupled to a flashlamp so that electrical current from the power supply may be supplied to the flashlamp, whereby at least one pulse of the limited wavelength spectrum radiation may be directed to a patient.

A fourth aspect of the invention is directed to dermatological treatment flashlamp device comprising a power supply and a handpiece. The power supply comprises a chopper circuit with an inductive filter element. The handpiece is operably coupled to the power supply and comprises: a housing comprising a housing interior and an aperture opening into the housing interior, the aperture having an aperture length; a flashlamp mounted within the housing interior and operably coupled to the power supply so that light from the flashlamp passes through the housing interior and out of the aperture for dermatological treatment of a patient; and a light-passage-restricting mechanism within the housing interior configured to permit radiation above about 590 nm to pass from the housing interior and out through the aperture. The flashlamp has a flashlamp arc length, said flashlamp arc length being oriented generally parallel to and being about equal to-about 20% longer than the aperture length.

A fifth aspect of the invention is directed to a method for enhancing the profile of radiation from a dermatological treatment device. A dermatological treatment flashlamp device, comprising a power supply and a handpiece, is selected. The handpiece is operably coupled to the power supply and comprises: a housing comprising a reflecting surface defining a housing interior extending to an aperture; a flashlamp mounted within the housing interior and operably coupled to the power supply so that light from the flashlamp passes through the housing interior and out of the aperture for dermatological treatment of a patient; and a skin-contacting, radiation-transmitting element covering the aperture. Opposed surface portions of said reflecting surface are configured to converge relative to one another towards the aperture so to enhance the divergence of radiation passing through the aperture. The outer surface of said skin-contacting, radiation-transmitting element is positioned to be spaced-apart from the reflecting surface at the aperture to allow divergent radiation to pass therethrough, resulting in a smoothly varying intensity profile.

A sixth aspect of the invention is directed to dermatological treatment flashlamp device comprising a power supply and a handpiece, operably coupled to the power supply. The handpiece comprises a housing comprising a reflecting surface defining a housing interior and extending to an aperture; a flashlamp mounted within the housing interior and operably coupled to the power supply so that light from the flashlamp passes through the housing interior and out of the aperture for dermatological treatment of a patient; and a skin-contacting, radiation-transmitting element covering the aperture. The reflecting surface has opposed surface portions converging relative to one another towards the aperture so to enhance the divergence of radiation passing through the aperture. The skin-contacting, radiation-transmitting element has an outer surface spaced-apart from the reflecting surface at the aperture to allow divergent radiation to pass therethrough, resulting in a smoothly varying intensity profile.

A seventh aspect of the invention is directed to method for causing localized, cosmetically-desirable pigmentation in a patient's skin. A power supply is operably coupled to a flashlamp of a dermatological treatment flashlight device so that electrical current from the power supply may be supplied to the flashlamp. The wavelength of the radiation to be directed to a patient is limited to a chosen wavelength spectrum to be within the UVA through UVB wavelength spectrum. At least one pulse of radiation is directed from the flashlamp to a chosen location on a patient's skin causing localized pigmentation at the chosen location.

An eighth aspect of the invention is directed to dermatological treatment flashlamp device, for causing localized, cosmetically-desirable pigmentation in a patient's skin, comprising a power supply and a handpiece, operably coupled to the power supply. The handpiece comprises a housing comprising a housing interior and an aperture opening into the housing interior, the aperture having an aperture length; a flashlamp mounted within the housing interior and operably coupled to the power supply so that light from the flashlamp passes through the housing interior and out of the aperture for dermatological treatment of a patient; and a light-passage-restricting mechanism within the housing interior configured to permit radiation within a chosen wavelength spectrum to pass from the housing interior and out through the aperture, the chosen wavelength spectrum being within the UVA through UVB wavelength spectrum so to cause localized pigmentation in a patient's skin.

A ninth aspect of the invention is directed to dermatological treatment flashlamp device comprising a power supply and a handpiece, operably coupled to the power supply. The handpiece comprises a housing comprising a housing interior and an aperture opening into the housing interior, the aperture having an aperture length; a flashlamp mounted within the housing interior and operably coupled to the power supply so that light from the flashlamp passes through the housing interior and out of the aperture for dermatological treatment of a patient; a handpiece cooling element, carried by the housing, cooling selected parts of the handpiece; and a notch-type light-passage-restricting mechanism within the housing interior configured to permit radiation within a chosen wavelength spectrum to pass from the housing interior and out through the aperture, the chosen wavelength spectrum being a shallow tissue-penetrating, strongly melanin-absorbing wavelength spectrum so to reduce the cooling load on the handpiece cooling element.

The use of a notch-type filter can result in a substantial reduction in heat load to tissue and handpiece components. The reduced heat load can result in the need to use less cooling power, an advantage which can be used to design a smaller and more ergonomic handpiece.

The use of a chopper circuit with an inductive filter allows for large current operation of flashlamps, independent of the lamp impedance. This is in contrast to both PFN and RDC operation, in which the impedance of the lamp determines the size of the current. Arbitrary control of the current, allowed through the use of the chopper circuit with inductive filter, means that very short length lamps can be used without limiting the amount of energy that can be discharged into the lamp. Short lamp lengths, in turn, can be used to increase the electrical-to-optical efficiency by matching the arc length to the desired optical aperture, thereby eliminating losses of light that occur if a long arc lamp is used with a smaller aperture. Given that effective dermatologic treatments put a limit of a few centimeters on the size of the treatment aperture, a clear overall efficiency advantage is seen in the current controlled operation of a relatively short arc lamp.

Other features and advantages of the invention will appear from the following description in which various embodiments have been described in detail in conjunction with the accompanying drawings.

### BRIEF DESCRIPTION OF THE DRAWINGS

FIG. 1 is a simplified schematic illustration of a dermatological treatment flashlamp assembly made according to the invention;

FIG. 1A is a simplified cross-sectional view of the components of the handpiece of FIG. 1;

FIG. 2 is an isometric view of the major operational components handpiece of FIG. 1 with portions broken away to show various elements;

FIG. 3 illustrates the components of FIG. 2 in an assembled condition as viewed from the skin-contacting surface;

FIG. 4 is a schematic diagram of the basic components of the controlled current source power supply of FIG. 1;

FIG. 5 is a plot of voltage vs. time for the capacitor of FIG. 4 and an associated series of constant voltage/current pulses;

FIG. 6 is a plot similar to that of FIG. 5 in which the pulses have different, in this case increasing, voltage/current amplitudes;

FIG. 7 is a simplified view illustrating the relationship between the flashlamp arc length and the aperture length for the handpiece of FIGS. 2 and 3;

FIG. 8 illustrates a typical flashlamp spot geometry for the handpiece of FIG. 1A; and

FIG. 9 illustrates a conventional flashlamp spot geometry.

### DETAILED DESCRIPTION OF THE INVENTION

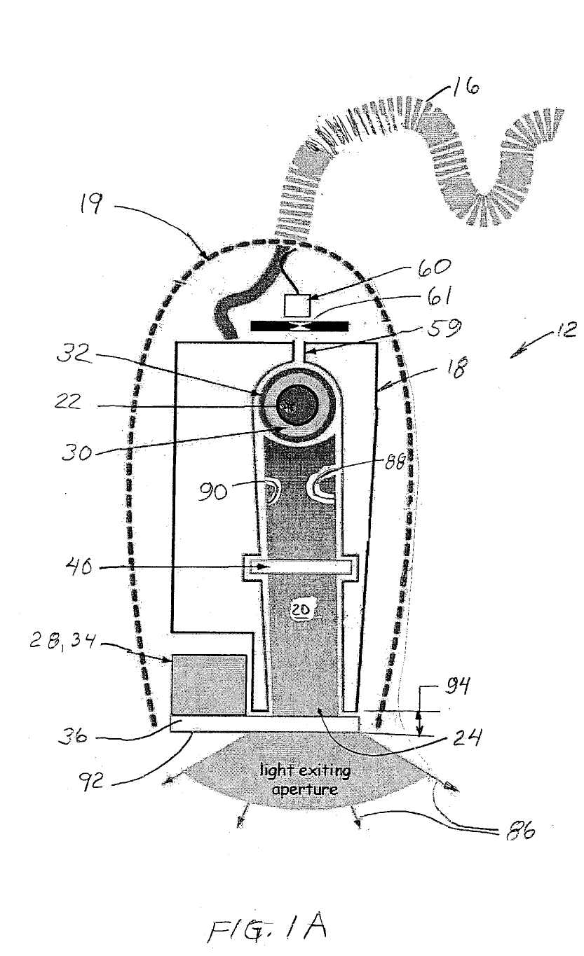

FIG. 1 illustrates a dermatological treatment flashlamp assembly 10 including a handpiece 12 connected to a power and control assembly 14 by a conduit 16. Handpiece 12, shown in FIGS. 1A, 2 and 3, comprises a housing 18, typically made of aluminum for its good thermal conductivity and its ability to have highly reflective surfaces, within a shell 19. Housing 18 defines a housing interior 20. A flashlamp 22 is mounted at one end of housing interior 20 with an aperture 24 formed at the opposite end of housing interior 20. Housing 18 defines a number of coolant channels 26 through which a coolant 27, typically distilled water, flows to remove heat from handpiece 12. In particular, coolant 27 passes through a heat sink 28 positioned adjacent to aperture 24 as well as through a gap 30 formed between flashlamp 22 and a UV-absorbing flowtube 32. Heat sink 28 is used to transfer heat away from the hot side of a thermoelectric device 34 sandwiched between a skin-contacting sapphire cover 36 and the heat sink.

Sapphire cover 36 substantially covers the outer end 38 of handpiece 12 and thus covers aperture 24 as well as thermoelectric device 34. The use of sapphire instead of, for example, glass for cover 36 is desirable because sapphire not only permits radiation from flashlamp 22 to pass through aperture 24 and to a patient's skin, but is an excellent heat conductor. This permits thermoelectric device 34 to more effectively cool skin-contacting cover 36 helping to prevent patient discomfort and, in some situations, unintended tissue damage. Coolant 27 passes along appropriate tubes, not shown, to and from handpiece 12 along conduit 16; electrical energy is supplied to flashlamp 22 along leads 42, 44 which also pass along conduit 16. Coolant 27 may be recycled using a heat exchange or may be replaced with fresh coolant.

Flowtube 32 blocks the passage of UV radiation, typically of wavelengths below about 350 nm, by absorbing the UV radiation and converting it into heat. The longer wavelength radiation passes into housing interior 20 and through a long wave pass filter 40 situated between flashlamp 22 and aperture 24. Filter 40 may be constructed to simply absorb shorter wavelengths or to reflect shorter wavelengths, or both absorb and reflect shorter wavelengths. It is currently preferred to provide filter 40 with a coating which reflects some shorter wavelengths to reduce the heat buildup within filter 40. This reflected radiation may be absorbed by the walls of housing 18, by flowtube 32 or by flashlamp 22, all of which are cooled by coolant 27. Together, flowtube 32 and filter 40 act as a notch-type light passage restricting mechanism, typically called a notch-type filter.

Three wavelength ranges, a long wavelength pass (such as about 580 or 590 or 600 or 610 nm and longer), a wide notch (590-850 nm) and a narrow notch (590-700 nm), are currently under consideration for use. In the notch filter embodiments, the heat load to tissue and to cover 36 can be reduced, while still producing the intended tissue effect, by a factor of about 2-10 depending on whether a narrow notch filter is used (with the heat load reduced by a factor of about 4 to 10), or a wide notch filter (with the heat load reduced by factor of about 2 to 5). This can result in the need for less cooling power, which can result in a smaller handpiece and more ergonomic design. A reduced heat load also creates a larger safety margin and can speed up treatment because there may be no need to stop to cool the window, as is often required with conventional devices. The reduced heat load may eliminate or reduce the need for use of a cooling gel.

Generally it may be desired to produce a broad wavelength band of, for example, 500-1100 nm for various dermatological treatments such as hair removal, small vessel or telangiectasia coagulation. However, in the treatment of pigmented lesions, such as solar lentigines, poikiloderma of Civette, melasma, hyperpigmentation, the purpose is to target relatively shallow pigments while avoiding strong absorption by hemoglobin in blood and vascular tissue, in which absorption peaks are located between 500 and 590 nm. Therefore, in such situations it may be desirable to limit the wavelength band to a shallow tissue-penetrating, but still strongly melanin-absorbing wavelength spectrum, such as 590-850 nm or 590-700 nm. Doing so helps to limit the depth of penetration of the radiation, which is quite shallow when treating pigmented lesions, thus reducing unnecessary tissue damage and patient discomfort. While a notch filter approach has several advantages in several situations, obtaining appropriately large flux levels using a notch filter approach creates practical difficulties. Therefore, a long wavelength pass embodiments may be preferred, especially for light skinned individuals or individuals with less melanin concentration in the targeted lesions.

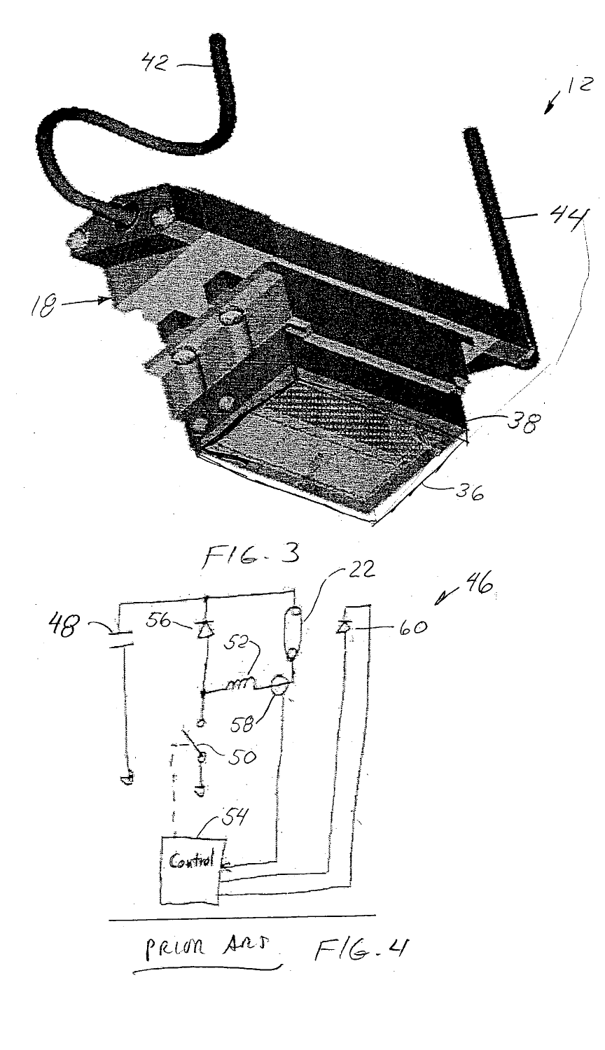

Assembly 40 also includes a power supply 46, shown schematically in FIG. 4. Power supply 46 is a controlled chopper circuit with an inductive filter element, operating in a pulse width modulated controlled current mode (in which the current is controlled and the voltage is not controlled). Power supply 46 is presently used to power a dermatological treatment laser device, sold by Altus Medical, Inc. of Burlingame Calif. as the CoolGlide® laser system for hair reduction and vascular indications. Alternatively, power supply 46 could be operated in a pulse width modulated controlled voltage mode (in which the voltage is controlled and the current is not controlled) or in a controlled power mode (in which the voltage and/or current are controlled in a manner resulting in controlled power). Energy storage capacitor 48 is charged to a level allowing the desired energy to be delivered without unacceptable lamp voltage droop at the desired current. Switch 50 is closed which ramps up current through lamp 22, inductor 52, and switch 50. When the appropriate current is reached, the control circuit 54 turns off the switch 50 and the current flow diverts to the diode 56. When the current flow decays to an appropriate level (typically 75% of the peak value) the control circuit again turns on the switch 50 and the cycle repeats until a pulse 57 is complete. This turning of switch 50 on and off during a single pulse 57 creates a slight ripple in pulse 57 as indicated in FIG. 5. The operation of assembly 40 in a controlled power mode refers to both the electrical energy delivered to lamp 22 and the resulting controlled optical power from lamp 22. Current sensor 58 and photodiode 60 are used independently or in concert to control the optical power delivered to skin. Photodiode 60, see FIG. 1A, may be placed at the top of housing 18 opposite a pair of apertures 59, and 61. The treatment waveform of the optical energy created by lamp 22 corresponds generally to the treatment waveform of the electrical energy delivered by power supply 46 to lamp 22.

Lamp life is a concern in high energy flashlamp systems. Instead of using a treatment waveform comprising one large pulse tens of milliseconds long, according to the present invention the power supply can modulate the lamp power in such a manner that the treatment waveform comprises many shorter, lower power pulses with small gaps between them. The gaps decrease the maximum thermal load and plasma discharge wall loading power by allowing the plasma to thermally relax between each shorter pulse. This reduced loading should result in longer lamp life. For example, instead of supplying a flashlamp with a treatment waveform comprising a single pulse 20 ms long, the flashlamp can be supplied with, for example, a treatment waveform comprising one or more of the following power pulse sequences: 8 power pulses each 2 ms long separated gaps approximately 0.6 ms long; 4 power pulses each 4 ms long separated gaps 0.75 ms long; 16 power pulses each 1 ms long separated gaps 0.25 ms long; 2 power pulses each 9 ms long separated gaps 2 ms long. In addition, a power pulse sequence may include power pulses of different durations separated by the same or different length gaps or of power pulses of equal durations separated by different length gaps.

In the present invention, the chopper circuit allows for current controlled operation of flashlamp 22. In current controlled operation, the impedance value of the lamp does not determine the amount of current that the driver can supply to the lamp. This has several consequences: A short flashlamp arc length 62 (or any other length) relative to the aperture length 64, see FIG. 7, can be used, thereby matching the desired treatment type and size, with attendant increase electrical-to-optical efficiency, a reduced stored energy requirement, and a more ergonomic handpiece design through a reduction in the required lamp dimensions. It is presently preferred that flashlamp arc length 62 be about equal to-about 20% longer than, and more preferably about 20% longer than, aperture length 64; that is, it is preferred that length 62 be approximately 20 percent longer than the treatment area width (typically 3 cm) in order to reduce end effects associated with the optical performance of the lamp arc in the region of the lamp electrodes. Arbitrary control of the lamp power waveform allows for a wide range of pulse amplitudes and widths. Arbitrary waveform generation is possible using power supply 46 but is not possible with PFN or RDC circuits. The range over which arbitrary lamp currents can be set with power supply 46 is typically 10:1, which can be selected within one pulse. RDC circuits can only set up for one current during a pulse and must accept the voltage and current droop associated with energy depletion of the storage capacitor. Capacitor voltage drops 66, 68, see FIGS. 5 and 6, do not affect output power as in the RDC circuit. This allows constant power pulses 57 to be generated with less stored energy. In a preferred design the capacitor voltage can drop by 50% before output power is affected at all. In a typical RDC design a capacitor voltage drop of 50% results in an 87% reduction in output peak power.

FIG. 6 illustrates a treatment waveform comprising an arbitrary pulse train, consisting of several pulses 70, 72, 74 of selected amplitudes, durations, intervals etc., to achieve the most effective treatment. In this example, successive pulses increase in amplitude in a potentially useful therapeutic treatment. Some pulse widths and constant or near-constant pulse amplitude (light intensity) combinations can be achieved with a controlled current source, such as power supply 46, that the PFN and RDC circuits in the non-notch filter versions either cannot achieve or require an impractical or uneconomical energy storage bank. Specifically, pulse widths >5 ms in combination with fluences in the >10 J/cm2 range are achievable with power supply 46 in the long wave pass (non-notch) embodiment but do not appear to be practical with these other technologies.

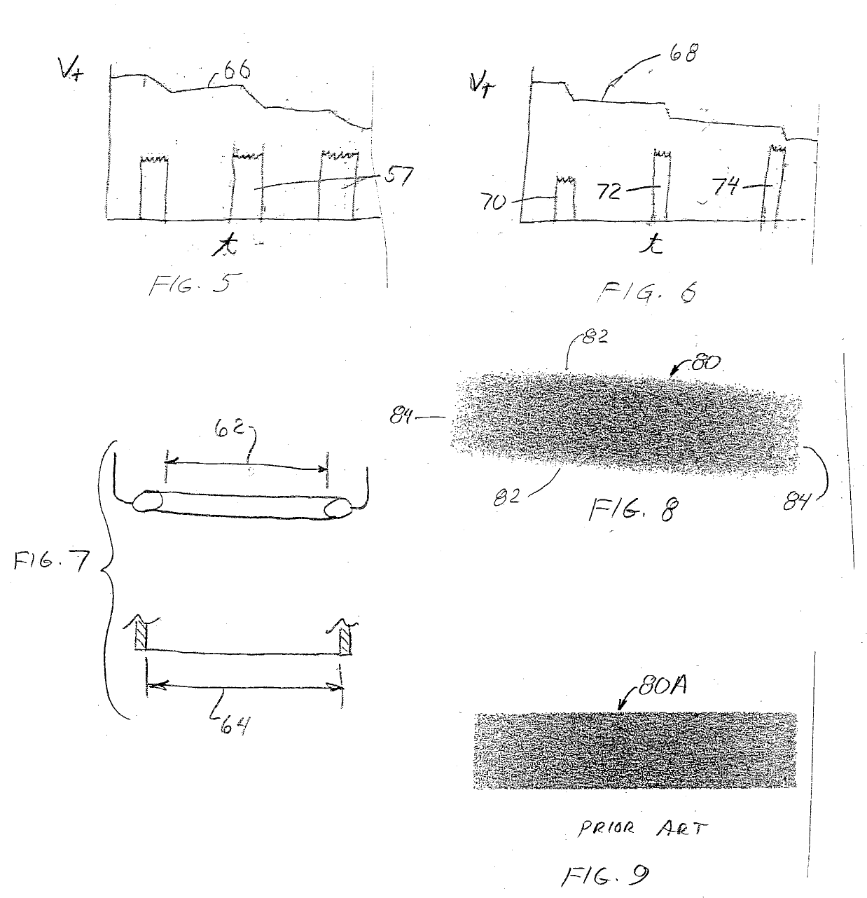

Aperture 24 of handpiece 12 is rectangular and housing interior 20 has a rectangular cross-sectional shape. They could, however, have other shapes as well. A typical flashlamp spot geometry 80 for handpiece 12 is shown in FIG. 8. Flashlamp spot geometry 80 is also generally rectangular with the longer sides 82 and shorter ends 84. The intensity profiles along both sides 82 and ends 84 are not sharp but rather are “feathered” with smoothly decreasing intensity. This is in contrast with conventional flashlamp optical intensity profiles, which typically have sharply delineated edges; one example of a conventional flashlamp spot geometry 80A, often termed a “top hat” geometry, is shown in FIG. 9. The feathered edges along sides 82 and ends 84 are created through a combination of an increase in the divergence of the light passing through aperture 24 and in the stand-off distance produced by the separation between the aperture 24 and the exit surface of the sapphire cover 36. This separation distance 94 is preferably about 1 to 5 mm, and more preferably about 2 to 4 mm. In one embodiment distance 94 is about 2.5 mm. The aperture 24 area, the divergence of the light, and the sapphire cover 36 thickness are chosen to allow for both a reasonably small spot geometry 80 and divergent, and therefore shallowly penetrating, optical intensity profile. Also, feathering of the edges may be useful for placement of treatment spots adjacent to one another without producing sharply contrasting treatment zones, which tend to be cosmetically unacceptable.

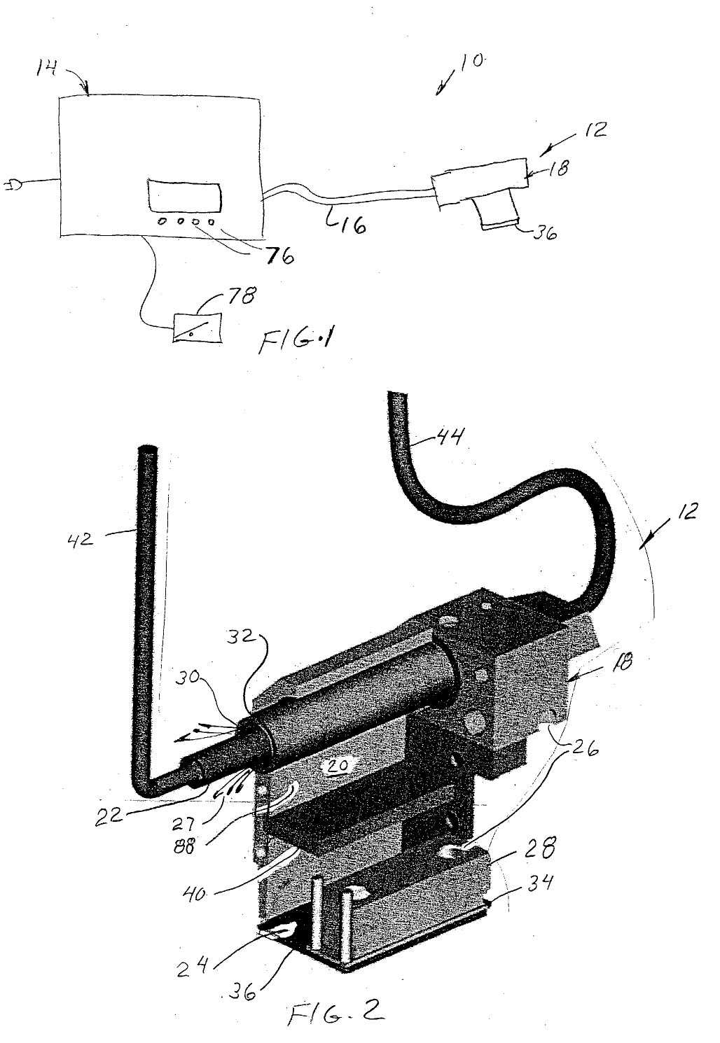

Melanin-containing pigmented lesions are in the epidermis or upper dermis so that it is very useful to limit the radiation to a shallow tissue-penetrating (aided by divergence), strongly melanin-absorbing wavelength spectrum. In the embodiment shown in FIGS. 1A and 2, this divergence, illustrated schematically by arrows 86, is enhanced. Reflective surfaces 88, 90 converge relative to another (by tapering downwardly and inwardly along their entire lengths) to enhance the divergence of the radiation along sides 82. Convergence may also be created by, for example, one or more of curving, stepping or tapering of at least a portion of at least one of the reflective surfaces.

Handpiece 12 may be selected according to the particular procedure to be conducted and the width (dimension) of the treatment area. Using controls 76 of assembly 14, the user may input one or more parameters, such as pulse width or widths, the optical fluence for each pulse, the period between pulses (which may be the same or different), the number of pulses delivered each time foot switch 78 is depressed, pulse shape. Power supply 46 of assembly 10 is preferably a chopper circuit with an inductive filter operating as a pulse width modulated current supply, and may also operate as a pulse width modulatedoptical power regulated supply. The waveform selected may have a generally constant current value equivalent to an optical fluence of at least about 1 J/cm2 (such as for narrow notch filter treatment of superficial lentigines in heavily pigmented skin) or at least about 4 J/cm2 (such as for lighter skin) or at least about 10 J/cm2 (such as for light lentigines in light skin). Also, a specific spectral range may influence the optical fluence so that, for example, the optical fluence for the narrow notch embodiment would typically not go above about 10 J/cm2 and the long wavelength pass embodiment would typically not be used below about 3 J/cm2. The waveform selected may also have a generally constant current value equivalent to an optical peak power producing a total fluence of between about 2 and 50 J/cm2. The waveform selected may have a generally constant current value equivalent to an optical fluence of at least about 10 J/cm2 with a pulse width of at least about 5 ms. The waveform may be selected to have a generally constant current value with a pulse width of about 1 to 300 ms, or about 5 to 50 ms, or about 10 to 30 ms. The waveform selected may have a generally constant current value and may be substantially independent of pulse width repetition rate. The settings will depend upon various factors including the type of treatment, the size of the lesion, the degree of pigmentation in the target lesion, the skin color or phototype of the patient, the location of the lesion, and the patient's pain threshold. Some or all of the operational parameters may be pre-set and not be user-settable. In particular, the bandwidth spectrum, such as 590-1100 nm, 590-850 nm, and 590-700 nm, will generally be fixed for a particular handpiece 12. However, it may be possible to construct handpiece 12 so that appropriate wavelength filters and reflectors may be changed by the user to change the wavelength of the output radiation. After the appropriate settings have been made, the flow of coolant 27 is actuated through the use of controls 76. Cover 36 of handpiece 12 is placed at the target site on the patient's skin, foot switch 78 is depressed, causing radiation to pass from flashlamp 22 through cover 36 at aperture 24, and the user begins moving handpiece 12 over the patient's skin. Coolant 27 keeps sapphire cover 36 from overheating during use while the radiation treats the pigmented lesion.

Another embodiment of the invention is directed to producing cosmetically desirable pigmentation in the skin in a spatially and temporally controlled manner. Melanin synthesis in melanocytes, or “melanogenesis”, refers to this process. Melanogenesis can take place as a photoprotective effect in response to UV radiation, and when it occurs in response to natural or artificial UV light, it is referred to as “tanning.”

A distinct phenomenon associated with true melanogenesis also occurs upon exposure to UV and visible light. “Immediate pigment darkening” (IPD) is a transient oxidative change to the state of existing melanin, occurs mostly in darker skin phototypes. The persistence of IPD is hours to days, and is not clinically useful in itself for treating pigmentation cosmetic problems. Strong IPD in dark skin phototypes indicates that longer-term (days to onset) melanogenesis will take place, and may serve as a clinical endpoint to pigmentation phototherapy [see Kollias N, Mallallay Y H, Al-Ajmi H, Baqer A, Johnson BE, Gonzales S. “Erythema and melanogenesis action spectra in heavily pigmented individuals as compared to fair-skinned Caucasians”, Photodermatol Photoimmunol Photomed 1996: 12: 183-188].

According to published melanogenesis action spectra [see Parrish J A, Jaenicke K F and Anderson R R. “Erythema and melanogenesis action spectra of normal human skin”, Photochem. Photobiol. Vol. 36. pp. 187-191, 1982], there is a strong dependence on wavelength, with the threshold dose rising rapidly as the wavelength increases from the end of the UVB (280-320 nm) into the UVA (320-400 nm). Beyond 400 nm, there is very little melanogenesis. The minimum melanogenic dose (MMD) to achieve/obtain threshold pigment induction is on the order of 100 J/cm2 for 365 nm, 1-10 J/cm2 for 315 nm, and 0.1 J/cm2 around 300 nm. The MMD is roughly independent of skin phototype. [Parrish, et al, 1982.]

One preferred embodiment of flashlamp 22 can delivery to skin a maximum pulse of light of fluence 30 J/cm2 (in a 20 ms pulse) in the 350-1100 band. That means that approximately 3 J/cm2 (in 20 ms) is available in the UVA and about 1.5 J/cm2 (in 20 ms) in the UVB. Since the minimum melanogenic dose (MMD) for UVB is falls between 0.1 and 1.0 J/cm2, a few pulses of appropriately filtered light from handpiece 12 would induce intermediate-term persistence melanogenesis (tanning) over the course of a few days post-treatment. In particular, a filter or filter set substituted for the epidermal pigment removal filter 40, having a transmission band between 290 and 320 nm could deliver to skin between 0.1 and 1.0 J/cm2 in a single 20 ms flash. One or more flashes could be directed to specific local areas of the skin at which increased pigmentation is desired. Masking agents, such as sunscreens or other physical barriers could be interposed between the light aperture and the skin to produce specific shapes or small areas of exposure (smaller than aperture 24 of handpiece 12).

Similarly, one could use UVA light by selecting another filter or filter set that allows light between 320 and 400 nm to be transmitted and delivered to the skin. The available fluences in this band are somewhat higher than in the UVB. As above, as much as 3 J/cm2 (in a 20 ms flash pulse) could be delivered with the preferred flashlamp 22. In this case, since the MMD is so much higher (as much as 100 J/cm2) many pulses would have to be delivered, potentially numbering into the hundreds of pulses. However, since these pulses could be produced by power supply 46 at as much as 0.5 Hz in this example, a particular treatment area could be exposed to the desired amount of UVA in as little as (100 shots)*(0.5 Hz)=200 seconds.

In the case of UVA treatments, the pulses would typically be delivered at a modest repetition rate to prevent any thermal effects or heat buildup. For UVA highest average power treatments, the average power loading would be approximately (3 J/cm2)(0.5 Hz)=1.5 W/cm2. Some conduction cooling of the sapphire window, and possibly the skin would likely be needed in this case. Sapphire cover 36 in combination with the existing temperature stabilizing thermoelectric device 34 can, for example, remove at least 10W average power from the skin plus cover 36. Other wavelength spectra, including continuous and discontinuous spectra over one or both of the UVA-UVB spectrum, may be desirable.

Several advantages exist when the invention is adapted for providing pigmentation of the skin, including (1) easy control of treatment areas, placement and doses, (2) ability to adapt a particular wavelength filtering handpiece to a particular treatment (3) confinement of UV exposure to superficial layers of the epidermis and dermis through the beam divergence (through the reflector geometry) (4) “feathering” of the light intensity pattern by a combination of divergence control and optical window standoff distance between the reflector aperture and the skin.

Modification and variation can be made to disclosed embodiments without departing from the subject of the invention is defined in the following claims.

Any and all patents, patent applications and printed publications referred to above are incorporated by reference.

What is claimed is:

1. A method for driving a dermatological treatment flashlamp comprising:
selecting a power supply comprising a chopper circuit with an inductive filter element;
selecting a desired treatment waveform;
supplying current from the power supply to the flashlamp to create the desired treatment waveform; and
directing at least one pulse of radiation from the flashlamp to a patient.

2. The method according to claim 1 wherein the power supply selecting step comprises selecting a power supply which operates in a pulse width modulated controlled power mode.

3. The method according to claim 1 wherein the power supply selecting step comprises selecting a power supply which operates in a pulse width modulated controlled current mode.

4. The method according to claim 1 wherein the waveform selecting step comprises selecting a waveform having a generally constant current value equivalent to an optical fluence of at least about 10 J/cm2.

5. The method according to claim 1 wherein the waveform selecting step comprises selecting a waveform having a generally constant current value equivalent to an optical fluence of at least about 4 J/cm2.

6. The method according to claim 1 wherein the waveform selecting step comprises selecting a waveform having a generally constant current value equivalent to an optical fluence of at least about 1 J/cm2.

7. The method according to claim 1 wherein the waveform selecting step comprises selecting a waveform having a generally constant current value with a pulse width of at least about 5 ms.

8. The method of claim 1 wherein the waveform selecting step comprises selecting a treatment waveform comprising a power pulse sequence, said power pulse sequence comprising a series of power pulses having at least one chosen duration and separated by gaps, said gaps having at least one chosen term.

9. The method according to claim 8 where the chosen duration is preferably about 1-10 ms.

10. The method according to claim 8 where the chosen term is preferably 100 us to 10 ms.

11. The method according to claim 1 wherein the waveform selecting step comprises selecting a waveform having a generally constant current value equivalent to an optical fluence of at least about 10 J/cm2 with a pulse width of at least about 5 ms.

12. The method according to claim 1 wherein the waveform selecting step comprises selecting a waveform having 8 power pulses each about 2 ms long separated gaps about 0.6 ms long.

13. The method according to claim 1 wherein the waveform selecting step comprises selecting a waveform having 4 power pulses each about 4 ms long separated gaps about 0.75 ms long.

14. The method according to claim 1 wherein the waveform selecting step comprises selecting a waveform having 16 power pulses each about 1 ms long separated gaps about 0.25 ms long.

15. The method according to claim 1 wherein the waveform selecting step comprises selecting a waveform having 2 power pulses each about 9 ms long separated gaps about 2 ms long.

16. The method according to claim 1 wherein the waveform selecting step comprises selecting a waveform having a generally constant current value with a pulse width of about 1 to 300 ms.

17. The method according to claim 1 wherein the waveform selecting step comprises selecting a waveform having a generally constant current value with a pulse width of about 5 to 50 ms.

18. The method according to claim 1 wherein the waveform selecting step comprises selecting a waveform having a generally constant current value with a pulse width of about 10 to 30 ms.

19. The method according to claim 1 wherein the waveform selecting step comprises selecting a waveform having a generally constant current value and being substantially independent of pulse width or repetition rate.

20. The method according to claim 1 wherein the waveform selecting step comprises selecting a waveform having a generally constant current value equivalent to an optical peak power producing a total fluence of between about 2 and 50 J/cm2.

21. The method according to claim 1 wherein the waveform selecting step comprises selecting a waveform having a current value substantially independent of pulse repetition rate.

22. The method according to claim 1 wherein the waveform selecting step comprises selecting a waveform having a current function shape producing a generally constant optical output.

23. The method according to claim 1 wherein the waveform selecting step comprises selecting a waveform having a current function shape producing an optical output having a desired shape.

24. The method according to claim 1 further comprising determining an expected type of treatment, and then selecting a flashlamp type according to the type of treatment.

25. The method according to claim 1 further comprising determining an expected width of a treatment area, and then selecting a flashlamp having a flashlamp arc length corresponding to the expected width.

26. The method according to claim 25 wherein the flashlamp selecting step is carried out so that the flashlamp arc length is about equal to about 20 percent longer than the expected width.

27. The method according to claim 25 wherein the flashlamp selecting step is carried out so that the flashlamp arc length is about 20 percent longer than the expected width.

28. The method according to claim 1 wherein the waveform selecting step comprises selecting a pulse train of a chosen set of varying amplitudes.

29. The method according to claim 1 further comprising limiting the wavelength of the radiation to be directed to a patient to a chosen wavelength spectrum above about 590 nm.

30. The method according to claim 1 further comprising:
limiting the wavelength of the radiation to be directed to a patient to a chosen wavelength spectrum; and
choosing the chosen wavelength spectrum to be a shallow tissue-penetrating, strongly melanin-absorbing wavelength spectrum.

31. The method according to claim 30 wherein the choosing step is carried out by choosing a wavelength spectrum of at most about 590 to 850 nm.

32. The method according to claim 30 wherein the choosing step is carried out by choosing a wavelength spectrum of at most about 590 to 700 nm.

33. The method according to claim 30 wherein the choosing step is carried out so that the chosen wavelength spectrum is a continuous wavelength spectrum.

34. The method according to claim 30 wherein the limiting step is carried out using a notch-type light passage wavelength restricting mechanism.

35. The method according to claim 34 wherein the limiting step is carried out using a notch-type filter.

36. The method according to claim 34 wherein the limiting step is carried out using a combination of a radiation-absorbing filter and a radiation reflector.

37. The method according to claim 1 further comprising:
cooling selecting parts of the handpiece;
limiting the wavelength of the radiation to be directed to a patient to a chosen wavelength spectrum; and
choosing the chosen wavelength spectrum to be a shallow tissue-penetrating, strongly melanin-absorbing wavelength spectrum so to reduce the cooling required in the cooling step.

38. A method for driving a pigmented lesion treatment flashlamp comprising:
selecting a power supply comprising a chopper circuit with an inductive filter element;
selecting a desired waveform having:
a current value equivalent to an optical fluence of at least about 4 J/cm²;
a pulse width of at least about 5 ms; and
a repetition rate from a single pulse to about 3 Hz;
supplying a flashlamp with electrical current from the power supply having the desired waveform;
cooling at least one part of the handpiece;
limiting the wavelength of the radiation to be directed to a patient to a wavelength spectrum of above about 590 nm so as to reduce the amount of cooling required in the cooling step; and
directing said limited wavelength spectrum radiation to a patient.

39. The method according to claim 38 wherein the limiting step is carried out by choosing a wavelength spectrum of at most about 590 to 700 nm.

40. The method according to claim 38 wherein the waveform selecting step is carried out so that the optical fluence is about 16-30 J/cm².

41. The method according to claim 38 wherein the waveform selecting step is carried out so that the pulse width is about 20-30 ms.

42. The method according to claim 38 wherein the waveform selecting step is carried out so that repetition rate is from a single pulse to about 1 Hz.

43. The method according to claim 38 wherein the waveform selecting step is carried out so that the pulse width is about 20-30 ms, that repetition rate is from a single pulse to about 1 Hz, and the optical fluence is about 16-30 J/cm².

44. A method for enhancing the operation of a dermatological treatment flashlamp device comprising:
selecting a power supply comprising a chopper circuit with an inductive filter element; and
operably coupling the power supply to a flashlamp of a dermatological treatment flashlight device so that electrical current from the power supply may be supplied to the flashlamp, whereby at least one pulse of radiation from the flashlamp may be directed to a patient.

45. The method according to claim 44 wherein the power supply selecting step comprises selecting a desired current waveform so that electrical current from the power supply having the desired waveform may be supplied to the flashlamp.

46. The method according to claim 45 wherein the current waveform selecting step comprises selecting a pulse train of a chosen set of varying amplitudes.

47. The method according to claim 45 wherein the current waveform selecting step comprises selecting a pulse train of a chosen set of fixed amplitudes.

48. The method according to claim 44 wherein the power supply selecting step comprises selecting a power supply which operates in a pulse width modulated controlled current mode.

49. The method according to claim 44 further comprising determining an expected type of treatment, and then selecting a flashlamp type according to the type of treatment.

50. The method according to claim 44 further comprising determining an expected width of a treatment area, and then selecting a flashlamp having a flashlamp arc length corresponding to the expected width.

51. The method according to claim 50 wherein the flashlamp selecting step is carried out so that the flashlamp arc length is generally about 20 percent longer than the expected width.

52. The method according to claim 44 further comprising limiting the wavelength of the radiation to be directed to a patient to a chosen wavelength spectrum above about 590 nm.

53. The method according to claim 44 further comprising:
limiting the wavelength of the radiation to be directed to a patient to a chosen wavelength spectrum; and
choosing the chosen wavelength spectrum to be a shallow tissue-penetrating, strongly melanin-absorbing wavelength spectrum.

54. The method according to claim 53 wherein the choosing step is carried out by choosing a wavelength spectrum of at most about 590 to 850 nm.

55. The method according to claim 53 wherein the choosing step is carried out by choosing a wavelength spectrum of at most about 590 to 700 nm.

56. A method for enhancing the operation of a pigmented lesion treatment flashlamp device comprising:
selecting a power supply comprising a chopper circuit with an inductive filter element;
determining a treatment area width;
selecting a flashlamp having a chosen flashlamp arc length, said flashlamp arc length being chosen to be about 20% longer than the treatment area width;
limiting the wavelength of the radiation to be directed to a patient to a wavelength spectrum of above about 590 nm.; and
operably coupling the power supply to a flashlamp so that electrical current from the power supply may be supplied to the flashlamp, whereby at least one pulse of the limited wavelength spectrum radiation may be directed to a patient.

57. The method according to claim 56 further comprising selecting a desired current waveform.

58. The method according to claim 57 wherein the current waveform selecting step comprises selecting a pulse train having a plurality of power pulses separated gaps.

59. A dermatological treatment flashlamp device comprising:
a power supply comprising a chopper circuit with an inductive filter element; and
a handpiece, operably coupled to the power supply, comprising:
a housing comprising a housing interior and an aperture opening into the housing interior, the aperture having an aperture length;
a flashlamp mounted within the housing interior and operably coupled to the power supply so that light from the flashlamp passes through the housing interior and out of the aperture for dermatological treatment of a patient;
the flashlamp having a flashlamp arc length, said flashlamp arc length being oriented generally parallel to and being about equal to-about 20% longer than the aperture length;
a thermally cooled surface adjacent to the aperture;
a skin-contacting, radiation-transmitting element covering the aperture and the thermally cooled surface; and
a light-passage-restricting mechanism within the housing interior configured to permit radiation above about 590 nm to pass from the housing interior and out through the aperture.

60. The device according to claim 59 wherein the power supply is constructed to supply the flashlamp with electrical current having the following characteristics: a pulse width of about 20-30 ms, a repetition rate of from a single pulse to about 1 Hz, and a current value equivalent to an optical fluence of about 16-30 J/cm².

61. A method for enhancing the profile of radiation from a dermatological treatment device comprising:
selecting a dermatological treatment flashlamp device comprising:
a power supply; and
a handpiece, operably coupled to the power supply, comprising:
a housing comprising a reflecting surface defining a housing interior, said reflecting surface extending to an aperture opening into the housing interior, said reflecting surface comprising opposed surface portions;
a flashlamp mounted within the housing interior and operably coupled to the power supply so that light from the flashlamp passes through the housing interior and out of the aperture for dermatological treatment of a patient;
a skin-contacting, radiation-transmitting element covering the aperture, said skin-contacting, radiation-transmitting element comprising an outer surface;
configuring the opposed surface portions of said reflecting surface to converge relative to one another towards the aperture so to enhance the divergence of radiation passing through the aperture; and
positioning the outer surface of said skin-contacting, radiation-transmitting element to be spaced-apart from the reflecting surface at the aperture by a chosen distance to allow divergent radiation to pass therethrough, resulting in a smoothly varying intensity profile.

62. The method according to claim 61 wherein the configuring step is carried out by inwardly tapering the opposed surface portions relative to one another.

63. The method according to claim 61 wherein the configuring step is carried out by inwardly tapering the opposed surface portions relative to one another at a generally constant rate.

64. The method according to claim 61 wherein the positioning step is carried out so that the chosen distance is about 1-5 mm.

65. The method according to claim 61 wherein the positioning step is carried out so that the chosen distance is about 2-4 mm.

66. The method according to claim 61 wherein the positioning step is carried out so that the chosen distance is about 2.5 mm.

67. A dermatological treatment flashlamp device comprising:
a power supply; and
a handpiece, operably coupled to the power supply, comprising:
a housing comprising a reflecting surface defining a housing interior, said reflecting surface extending to an aperture opening into the housing interior;
a flashlamp mounted within the housing interior and operably coupled to the power supply so that light from the flashlamp passes through the housing interior and out of the aperture for dermatological treatment of a patient;
a skin-contacting, radiation-transmitting element covering the aperture;
said reflecting surface comprising opposed surface portions converging relative to one another towards the aperture so to enhance the divergence of radiation passing through the aperture; and
said skin-contacting, radiation-transmitting element comprising an outer surface spaced-apart from the reflecting surface at the aperture to allow divergent radiation to pass therethrough, resulting in a smoothly varying intensity profile.

68. The device according to claim 67 wherein the opposed surface portions taper inwardly relative to one another.

69. The device according to claim 67 wherein the opposed surface portions taper inwardly relative to one another at a generally constant rate.

70. A method for causing localized, cosmetically-desirable pigmentation in a patient's skin, comprising:
operably coupling a power supply to a flashlamp of a dermatological treatment flashlight device so that electrical current from the power supply may be supplied to the flashlamp;
limiting the wavelength of the radiation to be directed to a patient to a chosen wavelength spectrum;
choosing the chosen wavelength spectrum to be within the UVA through UVB wavelength spectrum; and
directing at least one pulse of radiation from the flashlamp to a chosen location on a patient's skin causing localized pigmentation at the chosen location.

71. The method according to claim 70 further comprising determining an expected dimension of a treatment area, and then selecting a flashlamp having a flashlamp arc length corresponding to the expected dimension.

72. The method according to claim 71 wherein the flashlamp selecting step is carried out so that the flashlamp arc length is about 20% more than the expected dimension.

73. The method according to claim 70 further comprising selecting a power supply comprising a chopper circuit with an inductive filter element which operates in a pulse width modulated controlled current mode.

74. The method according to claim 70 wherein the choosing step is carried out by choosing the UVA wavelength spectrum.

75. The method according to claim 70 wherein the choosing step is carried out by choosing the UVB wavelength spectrum.

76. The method according to claim 70 wherein the operably coupling step is carried out with the flashlamp mounted within a handpiece.

77. A dermatological treatment flashlamp device for causing localized, cosmetically-desirable pigmentation in a patient's skin, comprising:
a power supply; and
a handpiece, operably coupled to the power supply, comprising:
a housing comprising a housing interior and an aperture opening into the housing interior, the aperture having an aperture length;
a flashlamp mounted within the housing interior and operably coupled to the power supply so that light from the flashlamp passes through the housing interior and out of the aperture for dermatological treatment of a patient; and
a light-passage-restricting mechanism within the housing interior configured to permit radiation within a chosen wavelength spectrum to pass from the housing interior and out through the aperture, the chosen wavelength spectrum being within the UVA through UVB wavelength spectrum so to cause localized pigmentation in a patient's skin.

78. The device according to claim 77 wherein the flashlamp has a flashlamp arc length, said flashlamp arc length being oriented generally parallel to and being substantially equal to the aperture length.

79. The device according to claim 77 wherein the power supply is a controlled current source power supply.

80. The device according to claim 77 wherein the power supply is a pulse width modulated controlled current source power supply.

81. The device according to claim 77 wherein the handpiece comprises a skin-contacting, radiation-transmitting element covering the aperture.

82. The device according to claim 81 wherein the radiation-transmitting element comprises sapphire.

83. The device according to claim 77 further comprising a handpiece cooling element, carried by the housing, cooling selected parts of the handpiece.

84. The device according to claim 83 wherein the handpiece cooling element comprises a thermally cooled surface adjacent to the aperture.

85. The device according to claim 83 wherein the handpiece cooling element comprises:
a thermoelectric cooler comprising a cooled surface, adjacent to the aperture, and a heated surface; and
a heat sink adjacent to the heated surface, the heat sink comprising a passageway for the passage of a coolant therethrough.

86. The device according to claim 83 wherein:
the handpiece cooling element comprises a thermally cooled surface adjacent to the aperture; and
the handpiece comprises a radiation-transmitting element covering the aperture and the thermally cooled surface.

87. The device according to claim 77 wherein the light-passage-restricting mechanism comprises a combination of a radiation filter and a reflective surface.

88. The device according to claim 77 wherein the chosen wavelength spectrum is within the UVA wavelength spectrum.

89. The device according to claim 77 wherein the chosen wavelength spectrum is within the UVB wavelength spectrum.

90. A dermatological treatment flashlamp device comprising:
a power supply; and
a handpiece, operably coupled to the power supply, comprising:
a housing comprising a housing interior and an aperture opening into the housing interior, the aperture having an aperture length;
a flashlamp mounted within the housing interior and operably coupled to the power supply so that light from the flashlamp passes through the housing interior and out of the aperture for dermatological treatment of a patient;
a handpiece cooling element, carried by the housing, cooling selected parts of the handpiece; and
a notch-type light-passage-restricting mechanism within the housing interior configured to permit radiation within a chosen wavelength spectrum to pass from the housing interior and out through the aperture, the chosen wavelength spectrum being a shallow tissue-penetrating, strongly melanin-absorbing wavelength spectrum so to reduce the cooling load on the handpiece cooling element.

91. The device according to claim 90 wherein the flashlamp has a flashlamp arc length, said flashlamp arc length being oriented generally parallel to and being substantially equal to the aperture length.

92. The device according to claim 90 wherein the power supply is a controlled current source power supply.

93. The device according to claim 90 wherein the power supply is a pulse width modulated controlled current source power supply.

94. The device according to claim 90 wherein the handpiece comprises a skin-contacting, radiation-transmitting element covering the aperture.

95. The device according to claim 94 wherein the radiation-transmitting element comprises sapphire.

96. The device according to claim 90 wherein the handpiece cooling element comprises a thermally cooled surface adjacent to the aperture.

97. The device according to claim 90 wherein the handpiece cooling element comprises:
a thermoelectric cooler comprising a cooled surface, adjacent to the aperture, and a heated surface; and
a heat sink adjacent to the heated surface, the heat sink comprising a passageway for the passage of a coolant therethrough.

98. The device according to claim 90 wherein:
the handpiece cooling element comprises a thermally cooled surface adjacent to the aperture; and the handpiece comprises a radiation-transmitting element covering the aperture and the thermally cooled surface.

99. The device according to claim 90 wherein the light-passage-restricting mechanism comprises a combination of a radiation filter and a reflective surface.

100. The device according to claim 90 wherein the chosen wavelength spectrum is at most about 590 to 850 nm.

101. The device according to claim 90 wherein the chosen wavelength spectrum is at most about 590 to 700 nm.
